(19) United States
(12) Patent Application Publication
(10) Pub. No.: US 2024/0409525 A1
(43) Pub. Date: Dec. 12, 2024

(54) NATURALLY AVAILABLE FRUIT-DERIVED COMPOUNDS TESTED TO DOCK WITH PRESENILIN-1 AND AMYLOID PRECURSOR PROTEIN

(71) Applicant: Subhajit Dasgupta, Charleston, SC (US)

(72) Inventor: Subhajit Dasgupta, Charleston, SC (US)

(21) Appl. No.: 18/207,440

(22) Filed: Jun. 8, 2023

(51) Int. Cl. C07D 311/30; C07D 213/04; C07D 309/18

(52) U.S. Cl. CPC C07D 311/30; C07D 213/04; C07D 309/18

(57) ABSTRACT

The computer guided digital platform screened out test compounds in a cell free system using Molecular Docking and Molecular Dynamics. The experimental outcomes demonstrate complex structure formation between the test compounds Rhamnetin, Rosyrane, N-retinylidene-N-retinylethanolamine, Hesperidin, Trihydroxybutyrate, Trihydroxyflavone, Arylaminoethylamide with human Presenilin- and Amyloid Precursor Protein (APP). The compounds also demonstrate the ability to bind with amyloid beta (1-42) peptide. Trihydroxyflavone and its family, and arylaminoethylamide can modulate neurotransmitter release as the test compounds bind with synaptic vesicular protein VAT-1.

### CROSS REFERENCES

The provisional patent file to USPTO is on characterization of fruit derived compounds under the family of Trihydroxyflavone, Retinoids and Stilbenes aims identification of specific binding of the test compounds with membrane bound protease enzyme Gamma Secretase (GSA) and its substrate amyloid beta precursor protein (APP). The literature search has been performed by using the key words: (1) Trihydroxyflavone and Gamma Secretase; (2) Trihydroxyflavone and Amyloid precursor protein; (3) Trihydroxyflavone and Alzheimer's disease; (4) Trihydroxyflavone and dementia; (5) Retinoid and Gamma Secretase; (6) Retinoid and Amyloid precursor protein; (7) Retinoid and Alzheimer's disease; (8) Retinoid and dementia; (9) Stilbene and Gamma Secretase; (10) Stilbene and Amyloid precursor protein; (11) Stilbene and Alzheimer's disease; (12) Stilbene and dementia. The literature search engines are NCBI PubMed and PubChem compound, USPTO Patent search, Google Scholar.

The literature search demonstrates a total of 12 published articles (excluding overlapping articles) in the NCBI PubMed under key words number 1 to 4 as mentioned above. The overall findings suggest beneficial effect of Trihydroxyflavone compounds for Alzheimer's disease management. The compound Apigenin and Baicalein are commonly found in published research for trihydroxyflavone.

None of these published articles demonstrate the following Trihydroxyflavone compounds with clear mechanism of action in Alzheimer disease brain. The proposed compounds as test drugs for modulating enzyme action of Gamma Secretase and altering conformation of substrate Amyloid beta precursor protein (APP) are presented herewith:
1A. Rhamnetin (PubChem CID: 5281691); 1B. Hesperetin (PubChem CID: 72281); 1C. Tricetin (PubChem CID: 5281701).

We found these compounds bind specifically with Gamma Secretase catalytic component Presenilin-1 and Amyloid beta precursor protein (APPB). Out of several Trihydroxyflavone compounds we selected these 1A, 1B and 1C compounds to test further to confirm as drugs to control Alzheimer's neurological abnormalities.

In addition, the literature search on key word combinations number 5 to 8 on Retinoid compounds, we found a total of 800 published records on retinoic acid derivative. Being a component of Vitamin-A, the mode of action of Retinoid is explored in the aspect of RAR/ROR and RXR action in peripheral tissues under inflammatory versus anti-inflammatory conditions. There are seven published articles showing the beneficial action of Retinoids on management of Alzheimer's disease. The mouse and rat models are used to draw inference on effect of retinoids in neurodegeneration. We include here our published article on protective effect of Retinoid derived from carrot extract to prevent apoptosis of hippocampus cells in vitro (Dasgupta S, Bandyopadhyay M, 2013).

As found, none of these published articles demonstrated mode of action of these compounds: 2A. N-retinylidene-N-retinylethanolamine (PubChem CID: 11007064); 2B: iso-A2E (9-cis) (PubChem CID: 131769871); 2C. 2-[4(3-phenypropyl)-pyridin-1-ium-1yl]ethanol; chloride (PubChem CID: 160724477); 2D. 1-[4-[(6-Tert-butyl pyridine-3-yl)methyl]piperazin-1-yl}-2-dimethy aminoethanol (PubChem CID: 163762267); 2E: iso-A2E (11-cis) (PubChem CID: 131769870) on gamma secretase and amyloid precursor protein (APP).

We select these compounds 2A, 2B, 2C, 2D and 2E as drug to test efficacy to confirm its action on lowering neuronal abnormalities in Alzheimer's brain.

The literature search for Stilbene derivatives demonstrated 630 published articles show extraction of stilbene compounds and its effect on lowering inflammation. There are 10 out of 630 articles also demonstrated in mice and rat models that stilbenes are useful therapeutic biphenolic compounds and compound resveratrol has beneficial effect on lowering Alzheimer's clinical condition. Earlier, we found resveratrol has the ability to protect hippocampus cells from paraquat-induced apoptosis in vitro. Though, detailed action of stilbene compounds on modification of Gamma Secretase and APP to lower amyloid beta production and Alzheimer's condition is not completely reported in any published articles.

We herewith propose four Stilbene derivatives to test their efficacy to modulate Gamma Secretase action and prevent degradation of APP:
3A. Rosyrane (CID: 108443); 3B: Alpha-phenyl cinnamic acid (CID: 700620); 3C: Resveratrol (CID: 25113755); 3D. 2-phenyl maleic acid (CID: 5851674).

The cross references are cited along with.
1. Akaishi T, Morimoto T, Shibao M, Watanabe S, Sakai-Kato K, Utsunomiya-Tate N, et al. Structural requirements for the flavonoid fisetin in inhibiting fibril formation of amyloid beta protein. Neurosci Lett. 2008; 444 (3): 280-5.
2. Akaishi T, Yamamoto S, Abe K. 3′,4′,7-Trihydroxyflavone Downregulates NO Production in LPS- or IFN-gamma-Activated MG6 Microglial Cells by Attenuating the JNK-STAT1 Pathway. Biol Pharm Bull. 2022; 45 (3): 301-8.
3. Arsalandeh F, Ahmadian S, Foolad F, Khodagholi F, Farimani M M, Shaerzadeh F. Beneficial Effect of Flavone Derivatives on Abeta-Induced Memory Deficit Is Mediated by Peroxisome Proliferator-Activated Receptor gamma Coactivator 1alpha: A Comparative Study. Int J Toxicol. 2015; 34 (3): 274-83.
4. Braidy N, Behzad S, Habtemariam S, Ahmed T, Daglia M, Nabavi S M, et al. Neuroprotective Effects of Citrus Fruit-Derived Flavonoids, Nobiletin and Tangeretin in Alzheimer's and Parkinson's Disease. CNS Neurol Disord Drug Targets. 2017; 16(4): 387-97.
5. Dasgupta S, Bandyopadhyay M. Neuroprotective mode of action of resveratrol in central nervous system. PharmaNutrition. 2013; 1(3):90-7.
6. Dourado N S, Souza C D S, de Almeida M M A, Bispo da Silva A, Dos Santos B L, Silva V D A, et al. Neuroimmunomodulatory and Neuroprotective Effects of the Flavonoid Apigenin in in vitro Models of Neuroinflammation Associated With Alzheimer's Disease. Front Aging Neurosci. 2020; 12:119.
7. Elyasi L, Rosenholm J M, Jesmi F, Jahanshahi M. The Antioxidative Effects of Picein and Its Neuroprotective Potential: A Review of the Literature. Molecules. 2022; 27(19).
8. Fanaee-Danesh E, Gali C C, Tadic J, Zandl-Lang M, Carmen Kober A, Agujetas V R, et al. Astaxanthin exerts protective effects similar to bexarotene in Alzheimer's disease by modulating amyloid-beta and cholesterol homeostasis in blood-brain barrier endothelial cells. Biochim Biophys Acta Mol Basis Dis. 2019; 1865 (9): 2224-45.
9. Gao D, Chen C, Huang R, Yang C C, Miao B B, Li L, et al. Tetrahydroxy Stilbene Glucoside Ameliorates Cognitive Impairments and Pathology in APP/PS1 Transgenic Mice. Curr Med Sci. 2021; 41(2):279-86.
10. Islam F, Nafady M H, Islam M R, Saha S, Rashid S, Akter A, et al. Resveratrol and neuroprotection: an insight into prospective therapeutic approaches against Alzheimer's disease from bench to bedside. Mol Neurobiol. 2022; 59(7):4384-404.
11. Kim H J, Jung Y S, Jung Y J, Kim O H, Oh B C. High-Phytate Diets Increase Amyloid beta Deposition and Apoptotic Neuronal Cell Death in a Rat Model. Nutrients. 2021; 13(12).
12. Kuntz M, Candela P, Saint-Pol J, Lamartiniere Y, Boucau M C, Sevin E, et al. Bexarotene Promotes Cholesterol Efflux and Restricts Apical-to-Basolateral Transport of Amyloid-beta Peptides in an In Vitro Model of the Human Blood-Brain Barrier. J Alzheimers Dis. 2015; 48(3):849-62.
13. Li Q, Li X, Tian B, Chen L. Protective effect of pterostilbene in a streptozotocin-induced mouse model of Alzheimer's disease by targeting monoamine oxidase B. J Appl Toxicol. 2022; 42(11):1777-86.
14. Li Y, Zhao J, Holscher C. Therapeutic Potential of Baicalein in Alzheimer's Disease and Parkinson's Disease. CNS Drugs. 2017; 31(8):639-52.
15. Liu Q S, Jiang H L, Wang Y, Wang L L, Zhang J X, He C H, et al. Total flavonoid extract from Dracoephalum moldavica L. attenuates beta-amyloid-induced toxicity through anti-amyloidogenesic and neurotrophic pathways. Life Sci. 2018; 193:214-25.
16. Manna P R, Reddy A P, Pradeepkiran J A, Kshirsagar S, Reddy P H. Regulation of retinoid mediated StAR transcription and steroidogenesis in hippocampal neuronal cells: Implications for StAR in protecting Alzheimer's disease. Biochim Biophys Acta Mol Basis Dis. 2022; 1869(2):166596.
17. Marsh D T, Das S, Ridell J, Smid S D. Structure-activity relationships for flavone interactions with amyloid beta reveal a novel anti-aggregatory and neuroprotective effect of 2′,3′,4′-trihydroxyflavone (2-D08). Bioorg Med Chem. 2017; 25(14):3827-34.
18. Meng-Zhen S, Ju L, Lan-Chun Z, Cai-Feng D, Shu-da Y, Hao-Fei Y, et al. Potential therapeutic use of plant flavonoids in AD and PD. Heliyon. 2022; 8(11):e11440.
19. Muhammad M S, Ayo J O, Danjuma N M, AbdulWahab A, Isa A S, Umar A H. Modulation of Memory and Neurochemical Changes by Resveratrol and Environmental Enrichment in Rodent Model of Alzheimer's Disease. Niger J Physiol Sci. 2022; 37(1):59-67.
20. Pouso M R, Cairrao E. Effect of retinoic acid on the neurovascular unit: A review. Brain Res Bull. 2022; 184:34-45.
21. Rana M, Cho H J, Arya H, Bhatt T K, Bhar K, Bhatt S, et al. Azo-Stilbene and Pyridine-Amine Hybrid Multifunctional Molecules to Target Metal-Mediated Neurotoxicity and Amyloid-beta Aggregation in Alzheimer's Disease. Inorg Chem. 2022; 61 (27): 10294-309.
22. Sakalauskas A, Ziaunys M, Snieckute R, Smirnovas V. Autoxidation Enhances Anti-Amyloid Potential of Flavone Derivatives. Antioxidants (Basel). 2021; 10(9).
23. Sato A, Tagai N, Ogino Y, Uozumi H, Kawakami S, Yamamoto T, et al. Passion fruit seed extract protects beta-amyloid-induced neuronal cell death in a differentiated human neuroblastoma SH-SY5Y cell model. Food Sci Nutr. 2022; 10(5):1461-8.
24. Wei Y D, Chen X X, Yang L J, Gao X R, Xia Q R, Qi C C, et al. Resveratrol ameliorates learning and memory impairments induced by bilateral hippocampal injection of streptozotocin in mice. Neurochem Int. 2022; 159:105385.
25. Xie J, Li X, Zhou Y, Wu J, Tan Y, Ma X, et al. Resveratrol Abrogates Hypoxia-Induced Up-Regulation of Exosomal Amyloid-beta Partially by Inhibiting CD147. Neurochem Res. 2019; 44(5):1113-26.
26. Youn K, Yu Y, Lee J, Jeong W S, Ho C T, Jun M. Polymethoxyflavones: Novel beta-Secretase (BACE1) Inhibitors from Citrus Peels. Nutrients. 2017; 9(9).
27. Zhao L, Wang J L, Liu R, Li X X, Li J F, Zhang L. Neuroprotective, anti-amyloidogenic and neurotrophic effects of apigenin in an Alzheimer's disease mouse model. Molecules. 2013; 18(8):9949-65.

### STATEMENT OF FEDERALLY SPONSORED RESEARCH OR DEVELOPMENT (IF ANY)

Not Available.

### NAMES OF THE PARTIES TO A JOINT RESEARCH AGREEMENT

Not applicable.

### REFERENCES TO A SEQUENCE LISTING

A Sequence Listing XML file is submitted as a separate part of disclosure.

An incorporation by reference statement identifies the following clauses: Name of the XML File: Seqlisting_Newcompounds
Date of Creation: Jul. 25, 2023
Size of the XML File (bytes): 6210 bytes

### BACKGROUND OF INVENTION

Alzheimer's disease is a complicated neurological dysfunction followed by neurodegenerative condition found mostly in elderly people at the age of sixties. However, initiation of the disease pathology starts from 40-50 years ago period. Loss of synaptic transmissions in cortex and midbrain regions gradually lead the patients to dementia. Since decades, it has been found that, formation of neuronal tangles with deposition of amyloid beta (1-42) and phospho-tau peptides in different anatomical areas of mid brain, amygdala and hind brain is the cause of Alzheimer's clinical outcomes-loss of learning ability and dementia.

In last ten years of drug discovery research on Alzheimer's disease could not provide successful strategy to cure the disease. Considering three of the most noticeable areas of Alzheimer's research: 1. To prevent amyloid beta (1-42) self-aggregation and deposition; 2. To prevent formation of phospho-tau and neurofibrillary tangle; 3. To improve synaptic transmission and delay severity of clinical outcome including dementia, extensive research is going on throughout the world. The limited success so far is due to lack of suitable model and a wide syndrome involving different areas of brain still warrants drug discovery research to overcome the existing challenges; to find new therapeutic compounds.

In the three abovementioned critical aspects, we continue the Alzheimer's drug discovery research with search for new compounds and test the physical properties of the compounds to bind with protease enzyme Gamma Secretase (GSA) and its substrate APP. The target is to modify GSA active site for APP to cleave thus inhibit generation of amyloid beta (1-42). Also, to bind with APP and prevent APP degradation. We screened efficacy of 12 new compounds as future drugs to prevent generation/aggregation of amyloid beta (1-42).

### BRIEF SUMMARY

The test compounds are screened for their mode of action on Gamma Secretase (GSA) and APP by formation of binding complex. The binding efficacy of the test compounds with human APP transmembrane proteins (PDB ID: 2LLM); identified APP sequence (PDB ID: APP_L) and human Presenilin-1 soluble component (PDB ID: 2KR6) has been determined by Molecular Docking and Molecular Dynamics using Schrodinger PyMol software. The structure based PDB files are constructed separately for each test drug compound which are shown in the figures. The results demonstrate effective binding of the whole structure of the test drugs Rhamnetin (CID 5281691), Rosyrane (CID 180443) and N-retinylidene-N-retinylethanolamine (CID: 11007064).

### BRIEF DESCRIPTION OF SEVERAL VIEWS OF DRAWINGS

The efficacy of the screened and identified test compounds to bind with Gamma Secretase enzyme components and Amyloid Precursor Protein (APP) to alter enzyme function to degrade APP has been studied on digital platform by using Molecular Docking and Molecular dynamics. In the first set, five test compounds are taken to test their action. A brief descriptions are presented below.

FIG. 1. Trihydroxyflavone compound Rhamnetin effectively binds with human Gamma Secretase Complex Presenilin-1 (SEQ ID4) and human Amyloid Precursor Protein transmembrane domain (SEQ ID3).

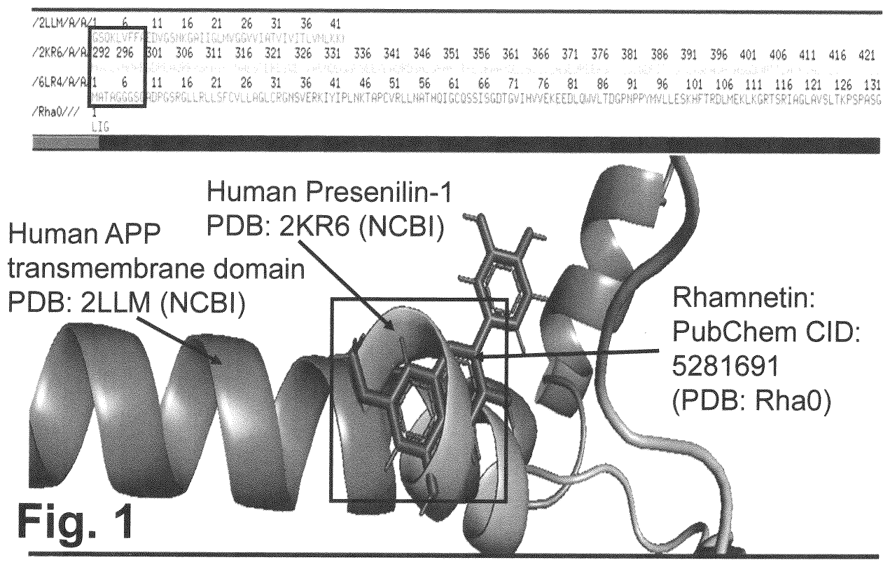

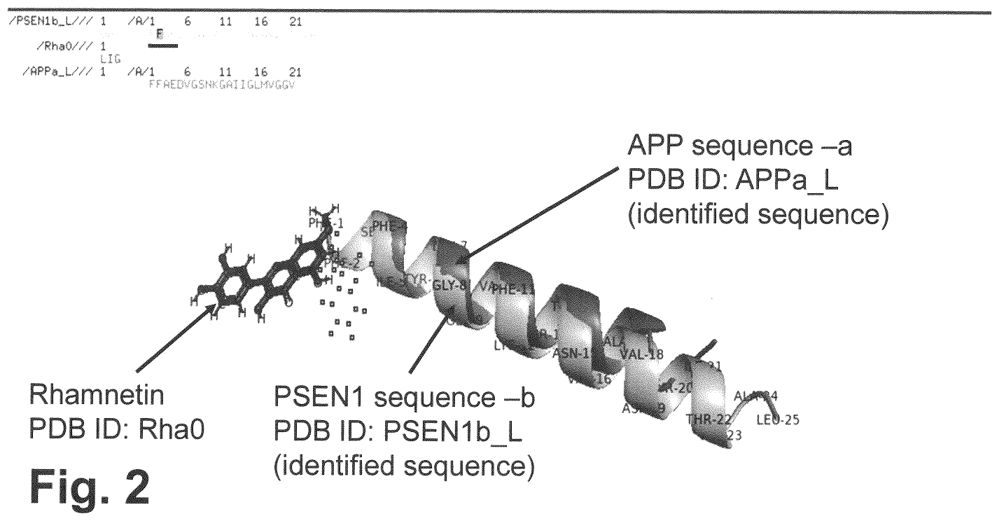

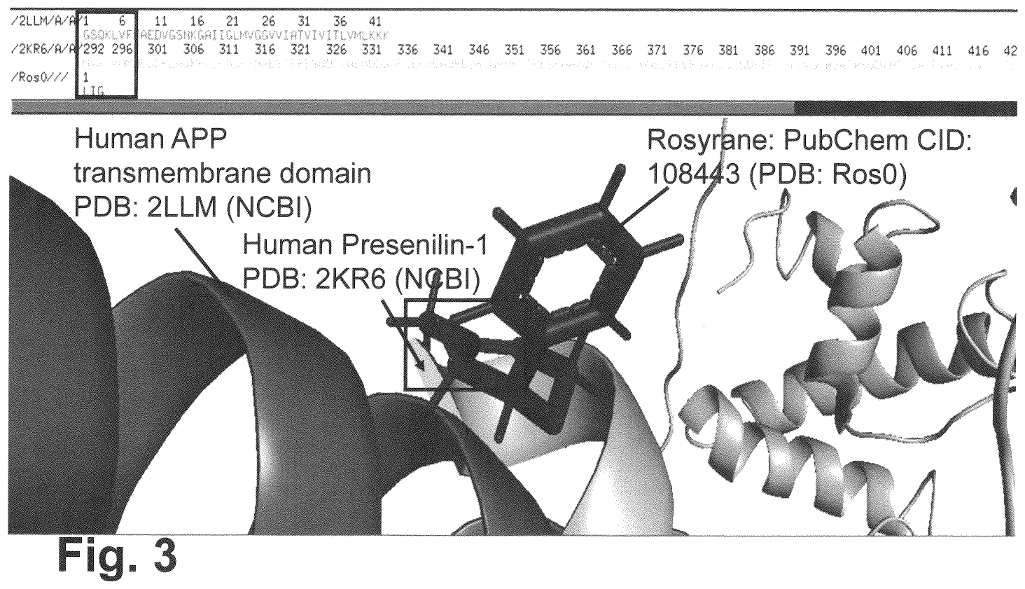

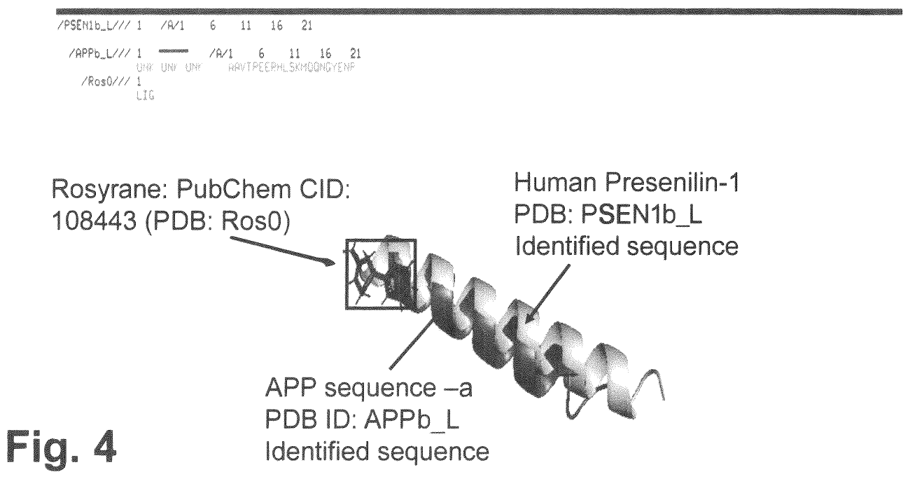

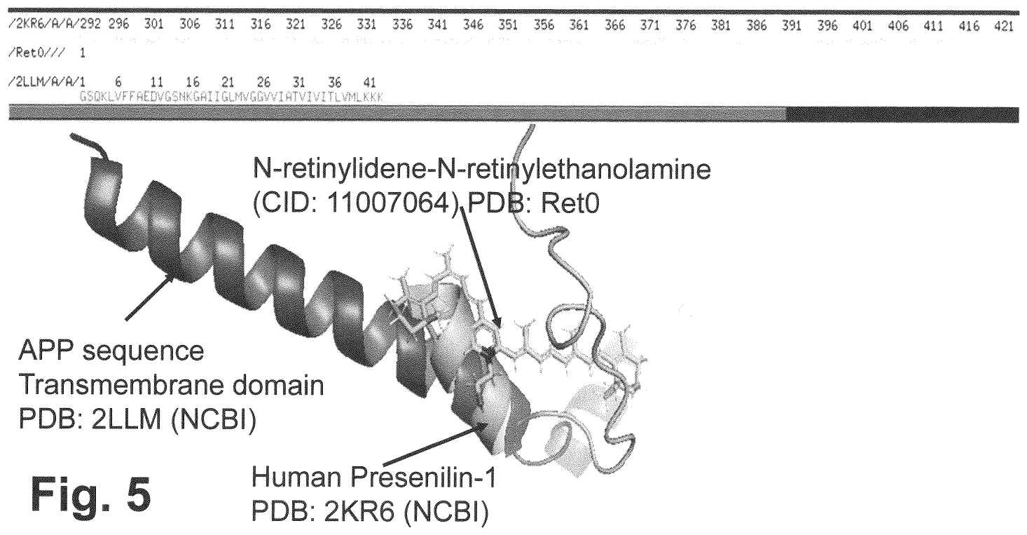

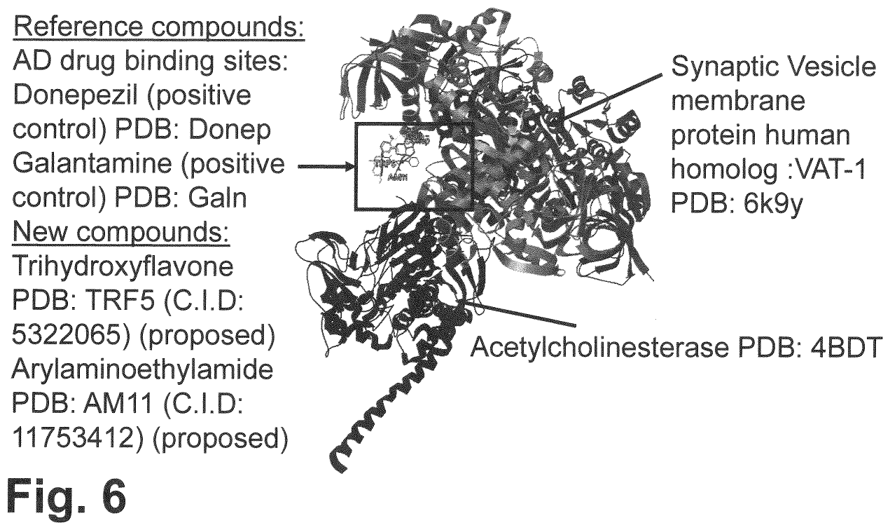

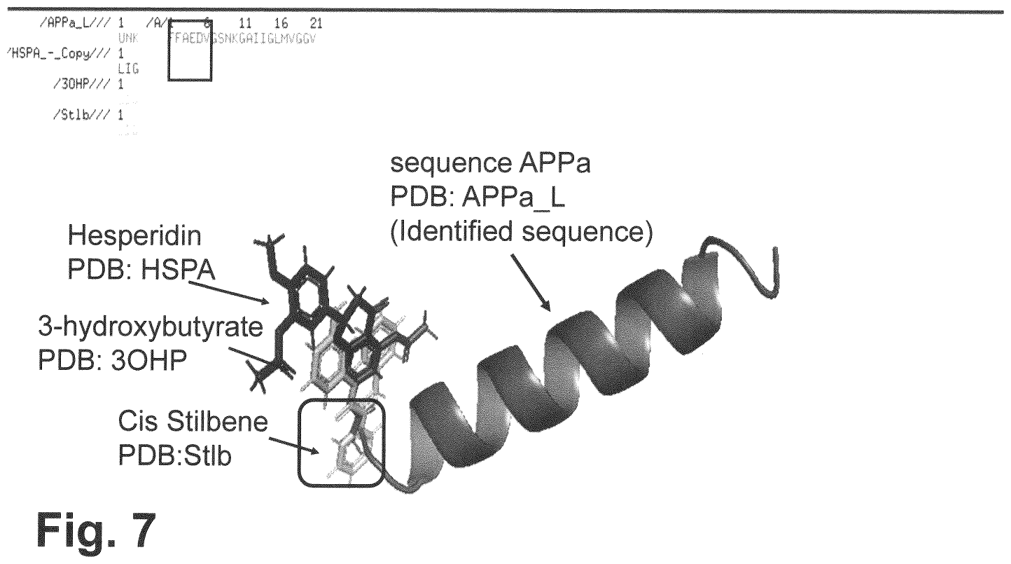

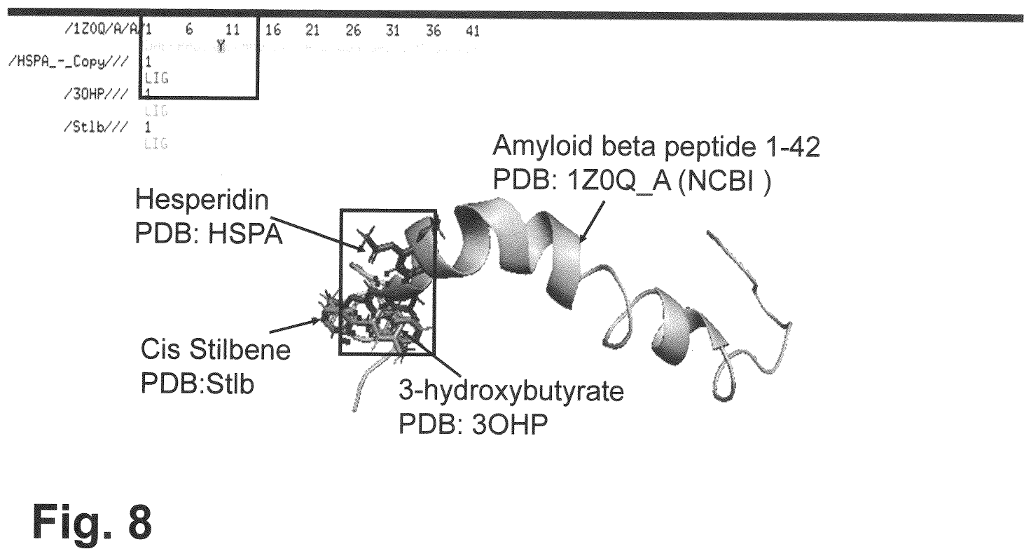

Rhamnetin (PubChem CID: 5281691) chemical structure has been sorted out by its canonical smiles conformation and converted to its corresponding protein database structure folder: Rha0. Under a digital setting with Schrodinger PyMol software, the three PDB files are allowed to align within each other interaction between Rhamnetin, GSA component Presenilin-1 (PDB: 2KR6) (SEQ ID4) and APP (PDB: 2LLM) (SEQ ID3). The amino acid sequences for Presenilin-1 and APP are programmed for alignment to test the binding efficacy of Rhamnetin. The experiment outcome demonstrates sequence specific binding of test drug Rhamnetin with human Presenilin-1 (2KR6) amino acid sequence 292-300 (inset box in the FIG. 1).

FIG. 2. Rhamnetin demonstrates binding with previously identified Presenilin-1 sequence PSEN1 sequence-b (PDB ID: PSEN1b_L; identified sequence) (SEQ ID1) and APP sequence-a (PDB ID: Appa_L) (SEQ ID2).

The figure demonstrates sequence specific binding of Rhamnetin (PDB: Rha0) with PSEN1 sequence-b (PDB: PSEN1b_L) (SEQ ID1). The APP sequence-a (PDB: APPa_L) (SEQ ID2) forms complex with PSEN1 sequence-b. The experimental methods are same as mentioned in the FIG. 1. The PSEN1 amino acid sequence-FFS forms bond with Rhamnetin.

FIG. 3. cis-Stilbene compound Rosyrane binds with human Presinilin-1 (SEQ ID4) and human APP transmembrane protein (SEQ ID3).

The chemical structure of cis-Stilbene compound Rosyrane (PubChem CID: 108443) has been sorted out by canonical smiles and converted into protein database file (PDB: Ros0) for computer-based experimentation purposes. Under the experimental setting using Schrodinger PyMol software, human Presenilin-1 (PDB: 2KR6) (SEQ ID4) is allowed to interact with human APP transmembrane domain (PDB: 2LLM) (SEQ ID3). The interaction of the structure complex with Rosyrane (PDB: Ros0) shows binding of the test drug compound Rosyrane with human Presenilin-1. The Rosyrane binding area on human APP (amino acid sequence: 1-6; GSQKLVF) and Presinilin-1 (amino acid sequence: 292-296: MVWLVNM) is shown in the FIG. 3 inset (in box).

FIG. 4. Rosyrane demonstrates binding complex formation with [identified] human Presenilin-1 (PDB ID: PSEN1b_L) (SEQ ID1) and identified APP sequence-b (PDB ID: APPb_L).

The test compound Rosyrane (PDB: Ros0) has been tested to form complex with human Presenilin-1 (PDB: 2KR6) and selected amino acid sequence of human APP APP sequence-b; PDB: APPb_L. The same experimental conditions are applied using Schrodinger PyMol software as mentioned in description of FIG. 1 and FIG. 3.

FIG. 5. Retinoid compound N-Retinylidene-N-Retinylethanolamine forms complex with human Presenilin-1 (SEQ ID4) and APP transmembrane domain (SEQ ID3).

Retinoid compound N-Retinylidene-N-Retinylethanolamine (PubChem CID: 11007064) chemical structure is selected by canonical smiles and converted to PDB file Ret0 for experimental purposes. The human Presenilin-1 (PDB 2KR6) (SEQ ID4) and APP sequence transmembrane domain (2LLM) (SEQ ID3) are aligned with test drug compound N-Retinylethanolamine (PDB Ret0) by computer guided Molecular Docking and Molecular Dynamics using Schrodinger PyMol software. The partial sequence of presenilin-1 (amino acid sequence 296-301: (MVWLVNM) interacts with APP sequence 1-41 (PDB: 2LLM) is presented in FIG. 5. The position of N-Retinylethanolamine structure complex formed with human presenilin-1 and APP is demonstrated in the figure.

Extended Experiments to Identify Binding Sites of Test Compounds with Receptors and Small Regulator Protein Molecules.

We extended our experiments to determine the ability of test compounds to bind receptors and small protein regulators for neurotransmitters. The rationale is to find out a clue on mode of action of the identified test compounds to regulate release of neurotransmitters. As a matter of fact, regulation of acetylcholine and gamma aminobutyric acid (GABA) release is the rate determining stage to overcome dementia syndrome in Alzheimer's disease and senility of elderly in general.

The Molecular docking experiments demonstrates the binding region(s) for the test compounds is/are located in a close vicinity which share with binding location for FDA approved available drugs: Donepezil and Galantamine. The identified test compounds Trihydroxyflavone (PDB: TRF5) and Arylaminoethylamide (AM11) are found to bind at the same location with the FDA approved reference compounds on synaptic vesicle membrane protein human homolog VAT-1 (PDB: 6k9y).

FIG. 6. Trihydroxyflavone and Arylaminoethylamide bind with synaptic vesicle membrane protein VAT-1.

The Molecular Docking experiment demonstrates binding site for proposed Alzheimer's disease drug test pound Trihydroxyflavone and Arylaminoethylamide on synaptic vesicle membrane protein VAT-1. The test compound binding site is close to binding site for FDA approved drug Donepezil and Galantamine. The synaptic vesicles transport neurotransmitter(s) from pre to post synaptic cleft of axons in neuron network in brain.

FIG. 7. Test compound Hesperetin (Hesperidin), trihydroxy phenyl butyrate and cis Stilbene bind with human amyloid precursor protein (identified sequence APPa) (SEQ ID2).

Molecular Docking experiment shows the test compounds Hesperidin (PDB: HSPA), Trihydroxybutyrate (PDB: 3OHP), cis Stilbene (PDB: Stlb) bind identified amino acid sequence: FFAED on amyloid precursor protein-a (APPa) (PDB: APPa_L) (SEQ ID2).

FIG. 8. Test compound Hesperetin, Trihydroxybutyrate and cis Stilbene bind with human amyloid beta 1-42 sequence (SEQ ID6).

Molecular Docking experiment demonstrates binding site: DAEFRHDSGYEV of the test compounds located in the N terminal site of amyloid beta 1-42 peptide (SEQ ID6).

### DETAIL DESCRIPTION

In order to detect specific binding site of the proposed five test drug compounds with human Gamma Secretase Component Presenilin-1 and substrate Amyloid precursor protein APP, the computer guided technology like Molecular Docking and Molecular Dynamics are used in the experiments. The FIG. 1 to FIG. 5 clearly demonstrate specific binding of the test compounds with corresponding receptor proteins.

The screened the new compounds presented in the NCBI PubChem site for modification of enzyme activity of human Gamma Secretase (GSA) and its substrate Amyloid Precursor Protein (APP) so the reaction system is inhibited to generate APP degradation product amyloid beta (1-42). The initially screened 12 compounds have been sorted out for further testing of their efficacy and binding affinity to GSA and APP or amyloid beta (1-42). Among these test compounds are selected-Rhamnetin, Rosyrane and N-retinylidene-N-retinylethanolamine for experiments.

The chemical structure canonical smiles are selected for each test compound from NCBI PubChem to create independent and separate protein database (PDB) file. The PDB files are used in Schrodinger PyMol software to generate digital format of these test compounds on a computer guided experimental platform which normalize all interactions between APP and Presenilin-1 to construct a stable complex structure (FIG. 1-5).

The test compounds Rhamnetin (PDB: Rha0), Rosyrane (PDB: Ros0) and N-retinylidene-N-retinylethanolamine (PDB: Ret0) are aligned with the APP-Presenilin-1 complex structure in Molecular docking and Molecular dynamics experimentations.

The aligned chemical structures with PyMol software generated (Display sequence mode for PDB) partial protein sequences are shown in the figures. The binding site of each test compound is demonstrated in the experiments (FIG. 1-5).

In the non-provisional application, we provided three more experimental evidence on binding location(s) of the proposed new AD drug-test compounds Trihydroxyflavone (PDB: TRF5), Arylaminoethylamide (PDB: AM11) (C.I.D: 11753412) on synaptic vesicle membrane protein VAT-1 (PDB: 6k9y) and acetylcholinesterase (PDB: 4BDT) (FIG. 6). The FIG. 6. Demonstrates VAT-1 binding location is close to similar with FDA approved currently available AD drug Donepezil and Galantamine.

The FIG. 7 demonstrates docking of the proposed AD drug test compounds Hesperidin (PDB: HSPA), Trihydroxybutyrate (PDB: 30HP) and cis Stilbene (PDB: Stlb) bind identified amino acid sequence of amyloid precursor protein (APP) (PDB: APPa_L) amino acid sequence: FFAEDV. We noted the binding of these proposed test compounds to other identified APP sequences: APPb and APPc.

The FIG. 8 demonstrates binding ability of these abovementioned three test compounds (reference FIG. 7) with amyloid beta (1-42) (abeta42) protein/peptide (PDB: 1Z0Q_A). The test compounds show the close alignment for binding domain at amino acid sequence: DAEFRHDSGYEV of Abeta42 (SEQ ID6).

1. The test compound Rhamnetin (PDB: Rha0) in its current chemical structure or functional group modified form retains the ability to bind protease enzyme Gamma Secretase (SEQ ID1; SEQ ID4) and Amyloid Precursor Protein (APP) (SEQ ID2; SEQ ID3).

2. The test compound Rosyrane (PDB: Ros0) in its current chemical structure or functional group modified form retains the ability to bind protease enzyme Gamma Secretase (Seq ID1; SEQ ID4) and Amyloid Precursor Protein (APP) (SEQ ID2; SEQ ID3).

3. The test compound N-retinylidene-N-retinylethanolamine (PDB: Ret0) in its current chemical structure or functional group modified form retains the ability to bind protease enzyme Gamma Secretase (Seq ID1; SEQ ID4) and Amyloid Precursor Protein (APP) (SEQ ID2; SEQ ID3).


SEQUENCE LISTING (6 sequences)

SEQ ID NO: 1 - protein, 25 aa, Homo sapiens
FFSFIYLGEVFKTYNVAVDYITVAL

SEQ ID NO: 2 - protein, 21 aa, Homo sapiens
FFAEDVGSNKGAIIGLMVGGV

SEQ ID NO: 3 - protein, 7 aa, Homo sapiens
GSQKLVF

SEQ ID NO: 4 - protein, 7 aa, Homo sapiens
MVWLVNM

SEQ ID NO: 5 - protein, 11 aa, Homo sapiens
MVWLVNMAEGD

SEQ ID NO: 6 - protein, 13 aa, Homo sapiens
DAEFRHDSGYEVH